(19) United States
(12) Patent Application Publication
(10) Pub. No.: US 2018/0119202 A1
(43) Pub. Date: May 3, 2018

(54) Proximity-enhanced nucleic acid-amplified protein detection

(71) Applicant: University of Washington, Seattle, WA (US)

(72) Inventors: Paul Yager, Seattle, WA (US); Joshua Bishop, Seattle, WA (US); Erin Heiniger, Seattle, WA (US)

(21) Appl. No.: 15/801,223

(22) Filed: Nov. 1, 2017

Related U.S. Application Data
(60) Provisional application No. 62/416,051, filed on Nov. 1, 2016.

(51) Int. Cl. C12Q 1/68; G01N 33/543

(52) U.S. Cl. CPC C12Q 1/6804; G01N 33/54306; C12Q 1/6818

(57) ABSTRACT

The present technology is related to methods and compositions for detecting, and optionally quantifying, one or more analytes of a sample using nucleic acids. In some embodiments, the methods include generating a complex of a plurality of peptides, an analyte, a first nucleic acid, and a second nucleic acid, each nucleic acid conjugated to a binder peptide. In addition, an immobilizer peptide can be immobilized to a substrate. If the binder peptides are bound to the analyte, the method further includes hybridizing a segment of the first nucleic acid to a segment of the second nucleic acid and amplifying the hybridized nucleic acids to generate a plurality of amplicons. Moreover, the generated amplicons indicate that one or more analytes has been detected. A number of generated amplicons can be analyzed to quantify one or more of the bound analytes.

### CROSS-REFERENCE TO RELATED APPLICATION(S)

The present application claims the benefit of U.S. Provisional Patent Application No. 62/416,051, filed Nov. 1, 2016, which is incorporated by reference herein in its entirety.

### STATEMENT REGARDING FEDERALLY SPONSORED RESEARCH

This invention was made with government support under Grant No. HDTRA1-16-C-0029, awarded by the Defense Threat Reduction Agency (DTRA). The government has certain rights in the invention.

### TECHNICAL FIELD

The present technology relates generally to nucleic acid-based methods for assaying one or more analytes within a biological sample. Many embodiments of the present technology relate to methods for detecting one or more nucleic acid analytes in the biological sample by proximity-enhanced nucleic acid amplification and associated compositions.

### BACKGROUND

Rapid diagnostic tests (RDTs) improve patient outcomes and decrease treatment costs for infectious diseases by providing information at the point of care with simplicity in a small disposable format. RDTs are low-cost and are useful for lower resource settings, where the burden of disease is often the greatest. However, currently available RDTs are more than one-hundred times less sensitive than nucleic acid amplification (“NAA”) based diagnostic tests. NAA-based biomarker tests are progressively replacing many peptide-based diagnostic tests, such as RDTs, currently used in clinical settings. Nevertheless, current NAA-based tests cannot detect peptides, and sample preparation to preserve and purify nucleic acids for NAA tests can be prohibitively time-consuming and expensive. In addition, NAA-based methods can be laborious and may involve false and/or unreadable results due to background noise. Therefore, biomarkers for certain diseases may not be detected, either because the RDTs are not sufficiently sensitive to detect low concentrations, or because the biomarkers are peptides and not detectible using NAA-based tests. A method for rapidly detecting biomarkers, using low-cost, simple, and reliable methods is needed.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a conceptual illustration of an analyte-binding complex associated with the method of proximity-enhanced nucleic acid amplified analyte detection in accordance with embodiments of the present technology.

FIG. 1B is a conceptual illustration of a portion of the analyte-binding complex illustrated in FIG. 1A in accordance with embodiments of the present technology.

FIG. 1C is a conceptual illustration of another portion of the analyte-binding complex illustrated in FIG. 1A in accordance with embodiments of the present technology.

FIG. 1D is a conceptual illustration of another portion of the analyte-binding complex illustrated in FIG. 1A in accordance with embodiments of the present technology.

FIG. 2 is a conceptual illustration of a first step of the method of proximity-enhanced nucleic acid amplified analyte detection in accordance with embodiments of the present technology.

FIG. 3 is a conceptual illustration of a second step of the method of proximity-enhanced nucleic acid amplified analyte detection in accordance with embodiments of the present technology.

FIG. 4 is a conceptual illustration of a third step of the method of proximity-enhanced nucleic acid amplified analyte detection in accordance with embodiments of the present technology.

FIG. 5 is a conceptual illustration of a fourth step of the method of proximity-enhanced nucleic acid amplified analyte detection in accordance with embodiments of the present technology.

FIG. 6 is a conceptual illustration of a fifth step of the method of proximity-enhanced nucleic acid amplified analyte detection in accordance with embodiments of the present technology.

FIG. 7 is a conceptual illustration of one or more nucleic acids associated with a method of proximity-enhanced nucleic acid amplified analyte detection in accordance with embodiments of the present technology.

FIGS. 8A and 8B are conceptual illustrations of another method of proximity-enhanced nucleic acid amplified analyte detection in accordance with embodiments of the present technology.

FIGS. 9A-9C are charts displaying amplification products generated by the method illustrated in FIGS. 8A and 8B, and generated by other methods of proximity-enhanced nucleic acid amplified analyte detection in accordance with embodiments of the present technology.

FIGS. 10A and 10B are charts displaying additional amplification products generated by the method illustrated in FIGS. 8A and 8B, and generated other methods of proximity-enhanced nucleic acid amplified analyte detection in accordance with embodiments of the present technology.

FIG. 11 is a chart displaying a relationship between fluorescence and amplification products generated by the method illustrated in FIGS. 8A and 8B, and other methods of proximity-enhanced nucleic acid amplified analyte detection in accordance with embodiments of the present technology.

FIGS. 12A-12C are charts displaying amplification products generated methods of proximity-enhanced nucleic acid amplified analyte detection in accordance with embodiments of the present technology.

### DETAILED DESCRIPTION

The following disclosure describes various methods for detecting and, optionally, quantifying one or more analytes of a sample using proximity-enhanced nucleic acid-based amplification and associated compositions. In some embodiments, for example, the methods include generating a complex of a plurality of peptides, an analyte, a first nucleic acid, and a second nucleic acid, each nucleic acid conjugated to a binder peptide. In some embodiments, an immobilizer peptide is immobilized to a substrate. If the binder peptides are bound to an analyte, the method further includes hybridizing a segment of the first nucleic acid to a segment of the second nucleic acid and amplifying the hybridized nucleic acids to generate a plurality of amplicons. In some embodiments, the generated amplicons indicate that one or more analytes have been detected. Further, in some embodiments, an amount of generated amplicons can be analyzed to quantify one or more of the bound analytes.

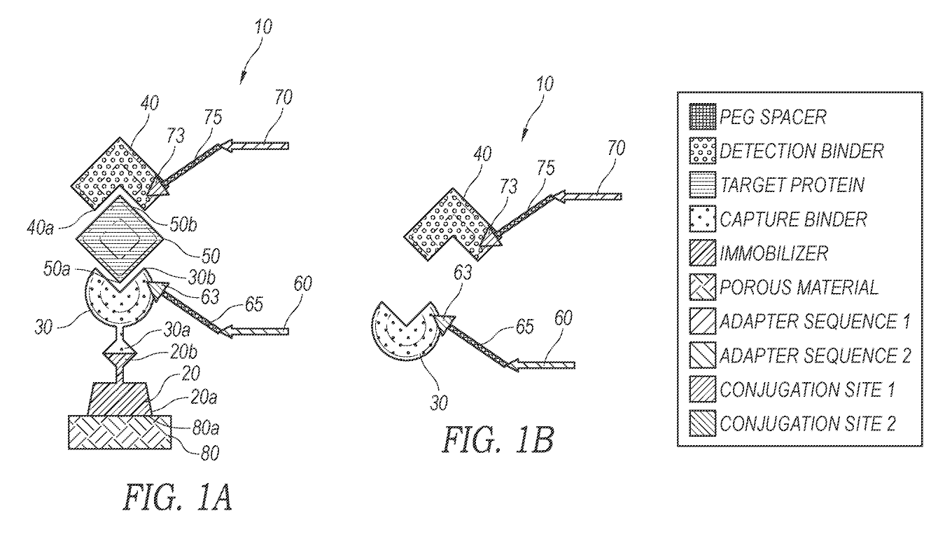

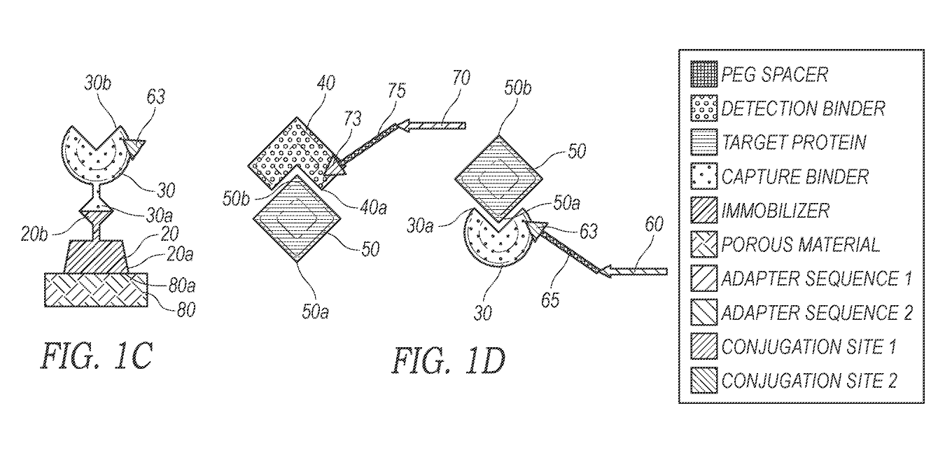

Certain details are set forth in the following description and FIGS. 1-12C to provide a thorough understanding of various embodiments of the disclosure. To avoid unnecessarily obscuring the description of the various embodiments of the disclosure, other details describing well-known methods, compositions, structures, and systems often associated with detection of analytes, nucleic acid amplification, and other methods associated with the present technology, are not set forth below or reflected in the Figures. Moreover, many of the details and features shown in the Figures are merely illustrative of particular embodiments of the disclosure. Accordingly, other embodiments can have other details and features without departing from the spirit and scope of the present disclosure. A person of ordinary skill in the relevant art will therefore understand that the present technology, which includes associated methods and compositions, may include other embodiments with additional elements or steps, and/or may include other embodiments without several of the features or steps shown and described below with reference to FIGS. 1-12C. Furthermore, various embodiments of the disclosure can include structures, features, and methods, other than those illustrated in the Figures and are expressly not limited to the structures, features, and methods, shown in the Figures.

### I. DEFINITIONS

As used herein, a “biological sample” can be any solid or fluid sample, living or dead, obtained from, excreted by, or secreted by any living or dead organism, including, without limitation, single-celled organisms, such as bacteria, yeast, protozoans, amoebas, multicellular organisms (such as plants or animals, including samples from a healthy or apparently healthy human subject or a human patient affected by a condition or disease to be diagnosed or investigated, such as tuberculosis) and/or soil. Biological samples can include one or more cells, proteins, nucleic acids, etc., as well as one or more buffers. Biological samples can be a liquid phase solution of cells or it may be a solid cell sample such as a cell pellet derived from a centrifugation procedure. As used herein, a “cell” or “cells” can refer to eukaryotic cells, prokaryotic cells, viruses, endospores or any combination thereof. Cells thus may include bacteria, bacterial spores, fungi, virus particles, single-celled eukaryotic organisms (e.g., protozoans, yeast, etc.), isolated or aggregated cells from multi-cellular organisms (e.g., primary cells, cultured cells, tissues, whole organisms, etc.), or any combination thereof, among others. The term “analyte,” as used herein, refers to a compound within a biological sample, including, but not limited to, a small molecule, a peptide, a polypeptide, a protein, a hormone, a nucleic acid, or a portion thereof. Analytes can also include biomarkers.

### II. Selected Embodiments of Methods of Proximity-Enhanced Nucleic Acid Amplified Analyte Detection and Associated Compositions

The present technology is directed to proximity-enhanced methods for detecting an analyte. As described further in the present disclosure, the analyte is detected upon binding to at least two binder peptides, a first binder peptide conjugated to a first nucleic acid and a second binder peptide conjugated to a second nucleic acid. In some embodiments, for example, the present proximity-enhanced methods include generating a plurality of amplicons from a hybridized segment of the first nucleic acid and the second nucleic acid, which hybridized upon detection of the analyte. In further embodiments, the plurality of amplicons are generated from the hybridized segment which forms when at least a first segment of the first nucleic acid and a second segment of the second nucleic acid are sufficiently proximate to one another, such as when the two binder peptides are bound to the analyte.

Proximity-enhanced methods for detecting the analyte include two labels, for example, the first nucleic acid and the second nucleic acid. By increasing proximity between the first nucleic acid and the second nucleic acid using peptide binders and an immobilizer, a relative concentration of the first nucleic acid and the second nucleic acid is increased. The increased relative concentration of the first nucleic acid and the second nucleic acid facilitates amplification of the first portion of the first nucleic acid and of the second portion of the second nucleic acid. In some embodiments, use of the first nucleic acid with the second nucleic acid increases sensitivity and stringency of the methods disclosed herein compared to detection methods including a single label.

FIG. 1A illustrates an immobilized composition 10 formed by the methods described herein. As shown in FIG. 1A, for example, the immobilized composition 10 is an immobilized analyte detection complex 10 and at least includes the immobilizer 20, the first binder peptide 30, and the second binder peptide 30. In some embodiments, the immobilizer includes a first zone 20a and a second zone 20b, with the first zone 20a configured to bind to a substrate 80 and the second zone 20b configured to bind to a first portion 30a of the first binder peptide 30. The substrate 20 can be a solid substrate. For example, the solid substrate can be a porous material, such as glass fiber, nitrocellulose, or a hybrid material (e.g., Fusion 5, a proprietary single layer matrix membrane commercially available from GE Healthcare). The methods can optionally include a tether (not shown) to post-translationally couple the immobilizer 20 to the first binding peptide 30. In addition, one or more components of the immobilized analyte detection complex 10 can be dried onto the substrate 80 and configured for storage until use.

As shown in FIG. 1A, the first binder peptide 30 further includes a second portion 30b configured to bind to a first region 50a of an analyte 50. In some embodiments, the second binder peptide 40 includes a first portion 40a configured to bind to a second region 50b of the analyte. For example, the first binder peptide 30 can be a capture binder configured to bind to the substrate 60, the immobilizer 20, or the tether, at a first portion of the capture binder. In addition, the first binder peptide 30 can be configured to bind to the analyte 50 at a second portion of the capture binder. In additional embodiments, the second binder peptide 40 is a detection binder configured to bind to the analyte 50. The first binder peptide 30 and the second binder peptide 40 may also form a peptide binder pair.

The first binder peptide 30 and/or the second binder peptide 40 may be peptides, polypeptides, antibodies, antibody fragments, aptamers, or a combination thereof. For example, the peptides, polypeptides, antibodies, antibody fragments, aptamers, or a combination thereof can be engineered peptides, designer peptides, synthetic peptides, or a combination thereof.

In some embodiments, one or more binder peptides or binder peptide pairs, has less than nanomolar (“nM”) affinities for binding to the analyte 50. For example, the one or more binder peptides or binder peptide pairs can have an equilibrium dissociation constant (K_{D}) of less than about 5E⁻⁷, about 1E⁻⁷, about 5E⁻⁸, about 1E⁻⁸, about 5E⁻⁹, about 1E⁻⁹, about 5E⁻¹⁰, about 1E¹⁰, about 5E⁻¹¹, about 1E⁻¹¹, about 5E⁻¹², about 1E⁻¹², about 5E⁻¹³, about 1E⁻¹³, about 5E⁻¹⁴, about 1E⁻¹⁴ about 5E⁻¹⁴ or less than about 1E⁻¹⁵ for binding to the analyte.

In other embodiments, one or more binder peptides or binder peptide pairs, has atomic level accuracy for binding to the analyte 50. For example, the one or more binder peptides or binder peptide pairs can have atomic level accuracy of less than about 10 Å, about 9.5 Å, about 9 Å, about 8.5 Å, about 8 Å, about 7.5 Å, about 7 Å, about 6.5 Å, about 6 Å, about 5.5 Å, about 5 Å, about 4.5 Å, about 4 Å, about 3.5 Å, about 3 Å, about 2.5 Å, about 2 Å, about 1.5 Å, about 1 Å, about 0.5 Å, or about 0.1 Å.

In further embodiments, one or more binder peptides or binder peptide pairs, has low off (K_{off}) values for binding to the analyte 50. For example, the one or more binder peptides or binder peptide pairs can have K_{off} value of less than about 0.05 K_{off} 1/Ms, about 0.01 K_{off} 1/Ms, about 0.005 K_{off} 1/Ms, about 0.001 K_{off} 1/Ms, about 0.0005 K_{off} 1/Ms, about 0.0001 K_{off} 1/Ms, about 5E⁻⁴ K_{off} 1/Ms, about 1E⁻⁴ K_{off} 1/Ms, about 5E⁻⁵ K_{off} 1/Ms, about 1E⁵ K_{off} 1/Ms, about 5E^{-6 K}_{off} 1/Ms, about 1E⁻⁶ K_{off} 1/Ms, about 5E⁻⁷ K_{off} 1/Ms, about 1E⁷ K_{off} 1/Ms, about 5E⁻⁸ K_{off} 1/Ms, about 1E⁸ K_{off} 1/Ms, about 5E⁻⁹ K_{off} 1/Ms, about 1E⁹ K_{off} 1/Ms, about 5E⁻¹⁰ K_{off} 1/Ms, about 1E⁻¹⁰ K_{off} 1/Ms, about 5E⁻¹¹ K_{off} 1/Ms, about 1E⁻¹¹ K_{off} 1/Ms, about 5E⁻¹² K_{off} 1/Ms, about 1E⁻¹² K_{off} 1/Ms, about 5E⁻¹³ K_{off} 1/Ms, or less than about 1E⁻¹⁴ K_{off} 1/Ms for binding to the analyte.

The present technology further includes methods for generating one or more binder peptides, such as binder peptide pairs that can be used with the proximity-enhanced methods described herein for detecting an analyte. Binder pairs can be designed using crystal structures of an analyte, such as a target analyte bound to one or more antibodies. In some embodiments, the one or more antibodies are neutralizing monoclonal antibodies. In other embodiments, however, the one or more antibodies are bound to one or more regions on the analyte. For example, the one or more regions can be different regions. In addition, the protein binder pairs bind to regions on the analyte that do not overlap. For example, the binder peptides of the pair are non-overlapping peptide binders.

In some embodiments, one or more binder peptides can be generated by adapting a commercially available antibody that binds to an analyte into a binder peptide. For example, one or more binder peptide pairs can be generated by adapting a pair of commercially available antibodies that bind to an analyte into a binder peptide pair. In these embodiments, each of the antibodies of the antibody pair bind to an epitope that is complimentary to another epitope on the same analyte. Furthermore, influenza nucleopeptide (“NP”) binder pairs can be designed by modifying a pair of commercially available antibodies that are specific to complementary epitopes of the influenza NP peptide.

In addition, one or more binder peptides, or binder peptide pairs, can also be generated by modifying an existing binder peptide, or binder peptide pairs, that bind(s) to an analyte of interest. For example, influenza hemagglutinin (“HA”) binder pairs can be designed by modifying HA stem binders and/or homo-trimeric HA head binders. In some embodiments, the HA stem binder can bind to either Group I influenza strains, Group II influenza strains, and/or can bind to other Group strains at a sialic acid binding site. In other embodiments, the homo-trimeric HA head binders can be engineered to bind to a sialic acid binding site.

One or more binder peptides, binder peptide pairs, or a portion thereof, of the present technology can be computationally designed. In other embodiments, one or more binder peptides, or binder peptide pairs, can also be generated by de novo computational design that bind an analyte of interest. For example, software such as the Rosetta Molecular Modeling suite (“Rosetta”), can be used to facilitate de novo computational design. The software can automate parallel de novo design of binder peptides, such as those that target different regions of the analyte. In addition, the regions can be, or can contain, one or more binding sites for the one or more binder peptides. These regions can be disposed upon a surface of the analyte. In some embodiments, a structure of the analyte or a portion thereof, or a structure of the region or a portion thereof, is known. For example, a known crystal structure. In other embodiments, a structure of the analyte or a portion thereof, or a structure of the region or a portion thereof, can be modeled using computer software, such as Rosetta.

The methods for generating one or more binder peptides can further include designing one or more binder peptides using a scaffold. In some embodiments, the scaffolds are mini-peptides. For example, the mini-peptides are stable mini-peptides having less than about 200 amino acids, less than about 190 amino acids, less than about 180 amino acids, less than about 170 amino acids, less than about 160 amino acids, less than about 150 amino acids, less than about 140 amino acids, less than about 130 amino acids, less than about 120 amino acids, less than about 110 amino acids, less than about 100 amino acids, less than about 90 amino acids, less than about 80 amino acids, less than about 70 amino acids, less than about 60 amino acids, less than about 50 amino acids, less than about 40 amino acids, less than about 30 amino acids, less than about 20 amino acids, less than about 10 amino acids, or less than about 5 amino acids.

In some embodiments, at least a portion of each of the binder peptides is resistant to proteolysis. For example, at least a portion of each of the binder peptides is not a substrate for an enzyme that cleaves a peptide into two or more separate and/or discrete portions. In other embodiments, however, at least a portion of each of the binder peptides is structurally stable at temperatures greater than about 80° C., about 85° C. , about 90° C., about 95° C., or about 100° C. For example, structurally stable binder peptides of the present technology maintain primary, secondary, tertiary, and, optionally, quaternary structures at temperatures of about 0° C., about 5° C., about 10° C., about 15° C., about 20° C., about 30° C., about 40° C., about 50° C., about 60° C., about 70° C., about 80° C., about 90° C., or at about 100° C. when at another temperature of about 0° C., about 5° C., about 10° C., about 15° C., about 20° C., about 30° C., about 40° C., about 50° C., about 60° C., about 70° C., about 80° C., about 90° C., or at about 100° C.

The methods for generating one or more binder peptides further include designing more than one binder peptide, such as two binder peptides, about ten binder peptides, about 20 binder peptides, about 30 binder peptides, about 40 binder peptides, about 50 binder peptides, about 100 binder peptides, about 500 binder peptides, about 1000 binder peptides, about 5000 binder peptides, about 10,000 binder peptides, about 25,000 binder peptides, about 50,000 binder peptides or more than about 50,000 binder peptides. In some embodiments, one or more genes encodes each binder peptide. A set of two or more binder peptides can be synthesized as a set of genes, such as open-reading frames. For example, for a set of about 50,000 binder peptides, a set of about 50,000 open reading frames can be synthesized. In addition, the set of open reading frames can be synthesized with chip-based oligonucleotides and compiled into a gene library.

In some embodiments, one or more gene libraries can be transformed into a target cell, such as yeast cells, and candidate binder peptides can be generated and screened for binding to the analyte. For example, to identify one or more binder peptides that bind to an analyte and that are stable, such as across temperatures disclosed herein and resistant to proteolysis, one or more libraries of yeast cells are generated from the transformed target cells. Furthermore, the one or more yeast libraries can be incubated at a range of temperatures, such as at about 10° C., about 15° C., about 20° C., about 25° C., about 30° C., about 35° C., about 40° C., about 45° C., about 50° C., about 55° C., about 60° C., about 65° C., about 70° C., about 75° C., or a combination thereof. In addition, the one or more yeast libraries can be incubated at a range of concentrations of one or more proteases, such as serum proteases. In some embodiments, the serum proteases are serine proteases, such as trypsin-like proteases, chymotrypsin-like proteases, thrombin-like proteases, elastase-like proteases, subtilisin-like proteases, or a combination thereof. For example, serum proteases can include one or more of the following, endoproteinase trypsin, chymotrypsin, endoproteinase ASP-N, endoproteinase Arg-C, endoproteinase Glu-C, endoproteinase Lys-C, pepsin, thermolysin, elastase, papain, proteinase k, subtilisin, clostripain, exopeptidase carboxypeptidase A, carboxypeptidase B, carboxypeptidase P, carboxypeptidase Y, cathepsin C, acylamino-acid-releasing enzyme, pyroglutamate aminopeptidase, or a combination thereof. Suitable concentrations of the one or more of the serum proteases can be within micromolar (μm), nanomolar (nm), picomolar (pm), or femtomolar (fm) ranges. Nucleic acid sequences of binder peptides which are stable across the temperatures disclosed herein and that are resistant to proteolysis are sequenced, for example, by next generation sequencing.

The methods can further include sorting, and optionally isolating, yeast cells expressing the stable and resistant binder peptide(s) from other yeast cells. The sorted yeast cells can be expanded to generate additional stable and resistant binder peptide(s). These stable and resistant binder peptides(s) can be tested for additional characteristics, such as binding with high affinity to the analyte, and in some embodiments, the analyte is labelled with a reporter. For example, the reporter can be a detectable marker, such as one or more quantum dots. In addition, the reporter can also be a fluorescent protein, such as a green fluorescent protein (GFP). Additional examples of suitable fluorescent detectable markers include, but are not limited to additional fluorescent proteins, e.g., enhanced-GFP (EGFP), Ds-Red (DsRed: Discosoma sp. red fluorescent protein (RFP); Bevis et al. (2002) Nat. Biotechnol. 20(11):83-87), yellow fluorescent protein (e.g., Venus), mCherry, mApple, blue-green emission tyrosine-derived chromophore (GEM), cyanofluorescent protein (e.g., Turquoise), variants thereof, and the like. Moreover, the detectable marker can be a colorless peptide that is a substrate for an enzyme which converts the colorless peptide into a marker detectible by color, such as fluorescence. These colorless peptides and enzymes can include rhodamine and horseradish peroxidase, respectively.

The binder peptide can also be subject to saturation site mutagenesis and labelling with a tag. For example, the label can be a His6 tag generated by an E. coli-based recombinant protein production technique and purified using immobilized metal chelate affinity (IMAC) purification. Furthermore, the binder peptide can also be tested for coincident target binding using biolayer interferometry and the binding mode and/or the structure of the protein binder is determined and/or verified by X-ray crystal structure determination.

In some embodiments, the one or more binder peptides are modified to increase binding affinity to a substrate. For example, the one or more binder peptides can be modified to have an equilibrium dissociation constant (K_{D}) of less than about 5E⁻⁷, about 1E⁻⁷, about 5E⁻⁸, about 1E⁻⁸, about 5E⁹, about 1E⁻⁹, about 5E⁻¹⁰, about 1E¹⁰, about 5E⁻¹¹, about 1E⁻¹¹, about 5E⁻¹², about 1E⁻¹², about 5E⁻¹³, about 1E⁻¹³, about 5E⁻¹⁴, about 1E⁻¹⁴, about 5E⁻¹⁴ or less than about 1E⁻¹⁵ for binding to the analyte. The substrate can be a solid substrate, such as a porous membrane.

As described in greater detail below, the proximity-enhanced methods for detecting an analyte include nucleic acid-based amplification. In some embodiments, one or more binder peptides are engineered for compatibility with nucleic acid-based amplification. For example, nucleic acid-based amplification can be proximity-enhanced isothermal strand displacement amplification (“PE-iSDA”). In addition, the one or more binder peptides can be modified for conjugation to one or more nucleic acids. For example, the one or more binder peptides can include the first binder peptide 30 and the second binder peptide 40 which can each be modified for conjugation to the first nucleic acid 60 and the second nucleic acid 70, respectively. More specifically, one or more binder peptides can be modified at a cysteine (“Cys”) residue. For example, the Cys residue can be modified using maleimide-thiol coupling chemistry. In these embodiments, the modified Cys residue can be a conjugation site, such as the first conjugation site 63 and/or the second conjugation site 73. The one or more binder peptides that have been engineered can further be transformed into target cells and processed, at least in part, as described above.

In some embodiments, the one or more engineered peptide binders are purified. For example, purification can be performed using a column, such as an affinity column (e.g., Ni-NTA Sepharose), which binds to the His6 tag. Purified engineered peptide can be verified using standard protein detection methods, such as SDS-PAGE and/or size exclusion chromatography. In other embodiments, the one or more engineered peptide binders can be complexed with one or more binders. For example, a binder peptide is an immobilizer homo-trimeric protein comprised of SpyCatcher quenched with SpyTag, a peptide aldolase homo-trimer, and monomeric streptavidin. In some embodiments, the immobilizer can bind to a plurality of substrates with one or more affinities. For example, the immobilizer can have dissociation constant (K_{D}) of less than about 5E⁻⁷, about 1E⁻⁷, about 5E⁻⁸, about 1E⁻⁸, about 5E⁻⁹, about 1E⁻⁹, about 5E⁻¹⁰, about 1E⁻¹⁰, about 5E⁻¹¹, about 1E⁻¹¹, about 5E⁻¹², about 1E⁻¹², about 5E⁻¹³, about 1E¹³, about 5E⁻¹⁴, about 1E⁻¹⁴, about 5E⁻¹⁴ or less than about 1E⁻¹⁵ for binding to one or more of the plurality of substrates. For example, the plurality of substrates is a fibrous paper (e.g., FF08HP, Fusion 5, and GF8964). Biolayer interferometry (BLI) experiments can also be performed to determine whether peptide binder pairs simultaneously and non-competitively bind to a target.

In some embodiments, each binder peptide of the binder pair can be conjugated to an oligonucleotide (“oligo”), the first nucleic acid 60, or the second nucleic acid 70. For example, the oligonucleotide can be an adapter oligonucleotide, a spacer oligonucleotide, or a combination thereof.

Referring again to FIG. 1A, the first nucleic acid 60 is conjugated to the first binder peptide 30 at a first conjugation site 63 via a first spacer 65 and the second nucleic acid 70 is conjugated to the second binder peptide 40 at a second conjugation site 73 via a second spacer 75. The conjugation sites 63/73 on each of the binder peptides in the pair can be aligned, such as geometrically aligned, to facilitate hybridization of the first nucleic acid 60 with the second nucleic acid 70 upon detection of the analyte 50. In some embodiments, the first spacer 65 and/or the second spacer 75 are polyethylene glycol (PEG) spacers.

Additionally, the present technology includes methods for generating an immobilized analyte detection complex 10. Such methods can include, for example, attaching the immobilizer 20 to the substrate 80, generating the complex 10 where the immobilizer is bound to the substrate 80, further comprising, binding the first portion 30a of the first binder peptide 30 to the first region 50a of the analyte 50, binding the first portion 40a of the second binder peptide 40 to the second region 50b of the analyte 50, and binding the section portion 30b of the first binder peptide 30 to the first zone 40a of the immobilizer peptide 40.

FIG. 1B illustrates a portion of the analyte-binding complex 10 of FIG. 1A in accordance with embodiments of the present technology. More specifically, FIG. 1B illustrates the first step where the first binder peptide 30 and the second binder peptide 40 are unbound to the immobilizer peptide 20. In some embodiments, the first nucleic acid 60 and the second nucleic acid 70 are conjugated to the first binder peptide 30 and the second binder peptide 40, respectively, before the first binder peptide 30 is bound to the immobilizer 20.

FIG. 1C illustrates another portion of the analyte-binding complex 10 of FIG. 1A in accordance with embodiments of the present technology. As illustrated in FIG. 1C, the first portion 30a of the first binder peptide 30 can bind to the first zone 20a of the immobilizer 20 before binding to the analyte 50 or conjugation to the first nucleic acid (not shown). In other embodiments, the first binder peptide 30 can be conjugated to the first nucleic acid 70 after binding to the second zone 20b of the immobilizer 20. In further embodiments, the first binder peptide 30 can bind to the analyte 50 before conjugation to the first nucleic acid 60 (not shown).

FIG. 1D illustrates yet another portion of the analyte-binding complex 10 of FIG. 1A in accordance with embodiments of the present technology. As illustrated in FIG. 1D, the first binder peptide 30 and/or the second binder peptide 40 are conjugated to the first nucleic acid 60 and the second nucleic acid 70, respectively, while each of the peptides are bound to the analyte 50. In some embodiments, for example, the first binder peptide 30 binds to the analyte 50 before the second binder peptide 40 binds to the analyte 50 (right panel). However, the second binder peptide 40 can also bind to the analyte 50 before the first binder peptide 30 binds to the analyte 50 (left panel). As illustrated, the first nucleic acid 60 is conjugated to the first binder peptide 30 via the first spacer 65 at the first conjugation site 63. In addition, the second nucleic acid 70 is conjugated to the second binder peptide 40 by the second spacer 75 at the second conjugation site 73.

The methods can also comprise detecting an analyte in a sample, such as a protein, by attaching the immobilizer peptide 20 to the substrate 80 and generating the complex 10 at the immobilizer peptide 20. In some embodiments, the complex 10 includes the analyte 50, the first binder peptide 30 bound to the immobilizer peptide 20 and the second binder peptide 40. In addition, methods of the present technology can also include iSDA. For example, with iSDA, the first nucleic acid 60 is a first template and the second nucleic acid 70 is a second template. The first template and the second template can also each comprise a portion of a probe binding site and a primer binding site (not shown). The method further includes detecting one or more of the plurality of amplicons generated by iSDA.

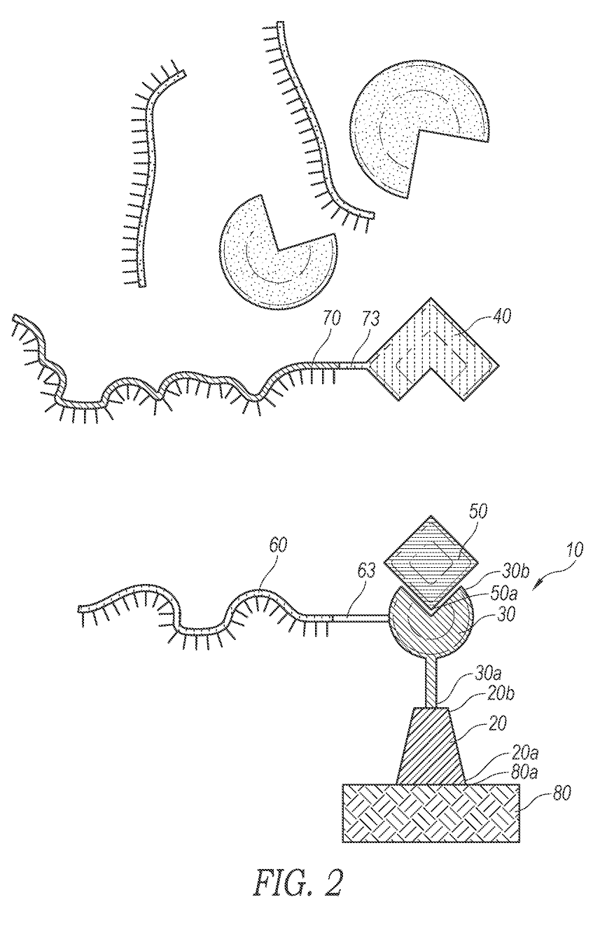

FIG. 2 illustrates a first step of the method of proximity-enhanced nucleic acid amplified analyte detection in accordance with embodiments of the present technology. As shown in FIG. 2, the disclosed method includes attaching the first zone 20a of the immobilizer 20 to the substrate 80 and the second zone 20b of the immobilizer 20 to the first portion 30a of the first binder peptide 30. In some embodiments, the second portion 30b of the first binder peptide 30 is bound to the first region 50a of the analyte 50. In addition, the first nucleic acid 60 is conjugated to the first binder peptide 30 via the first spacer 63. While illustrated in the analyte 50 unbound configuration, the second binder peptide 40 is conjugated to the second nucleic acid 70 via the second spacer 73 and, when bound to the analyte 50, a hybridization event occurs between the first portion of the first nucleic acid 60 and the second portion of the second nucleic acid 70 (not shown).

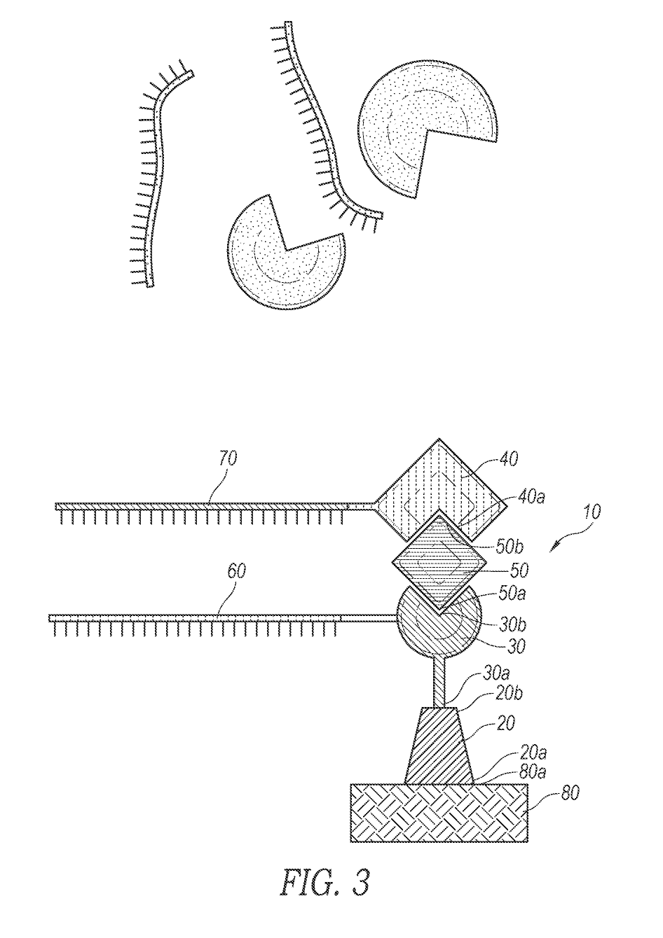

FIG. 3 illustrates a second step of the method of proximity-enhanced nucleic acid amplified analyte detection in accordance with embodiments of the present technology. As illustrated in FIG. 3, the second binder peptide 40 is bound to the second region 50b of the analyte 50. In some embodiments, the second nucleic acid 70 is conjugated to the second binder peptide 40. The location of the first conjugation site 63 and the second conjugation site 73 on each of the binder peptides can be selected based on the analyte of interest. For example, selection criteria for the location(s) include, but are not limited to, distance between the first and second nucleic acid upon conjugation to the respective binder peptides following analyte 50 binding, dimensions of the first binder peptide 30 and the second binder peptide 40, dimensions of the immobilizer 20, location of the first region 50a on the analyte 50, location of the second region 50b on the analyte 50, and combinations thereof.

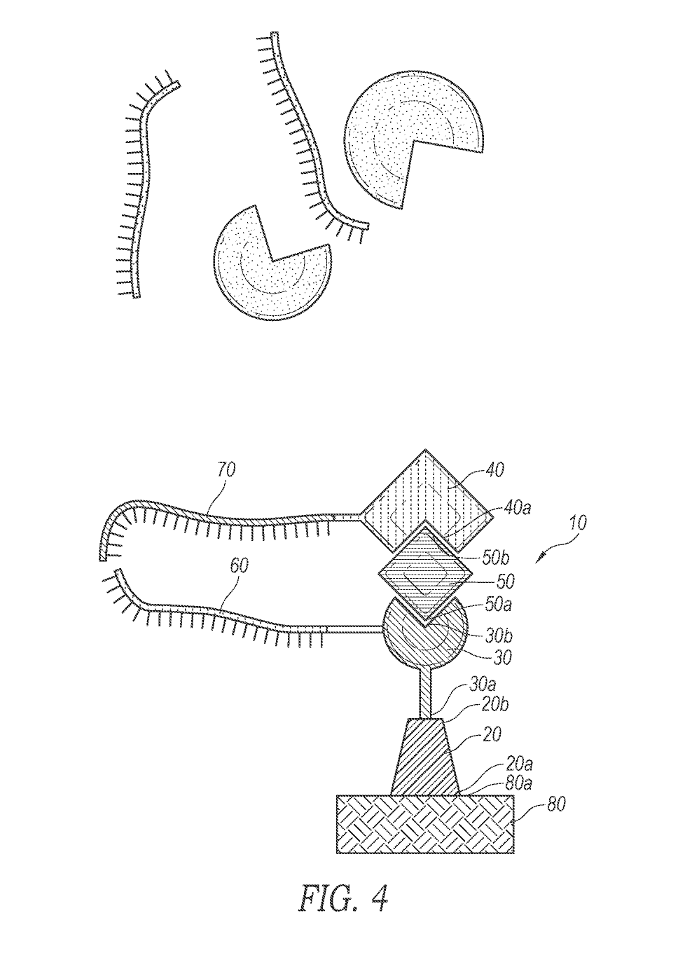

FIG. 4 illustrates a third step of the method of proximity-enhanced nucleic acid amplified analyte detection in accordance with embodiments of the present technology. As illustrated, the locations of the conjugation site bring the first nucleic acid 60 and the second nucleic acid 70 within proximity of one another. By specifying the location of the first conjugation site and/or second conjugation site 63/73, the first nucleic acid 60 and the second nucleic acid 70 can be a selected distance and/or position relative to one another. For example, the increased proximity enhances signal and/or reduces instances of non-specific amplification.

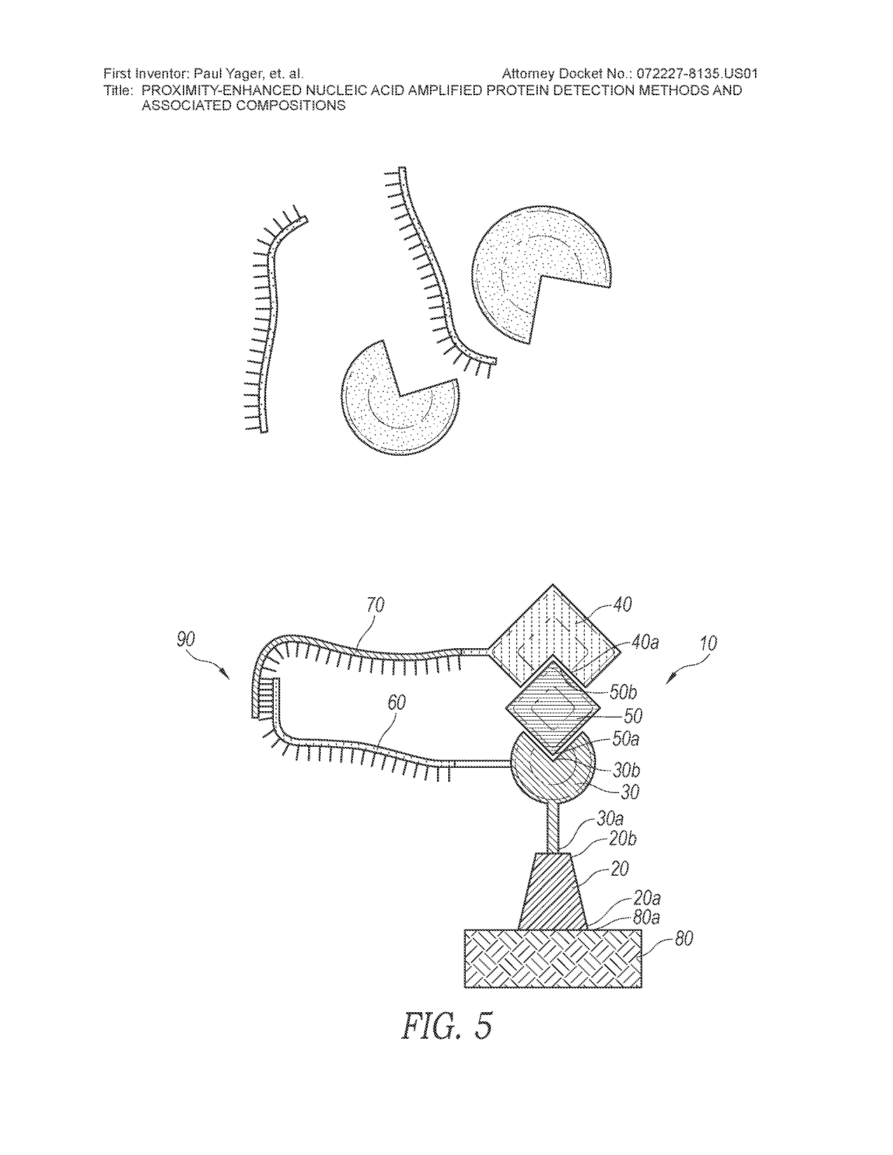

FIG. 5 illustrates a fourth step of the method of proximity-enhanced nucleic acid amplified analyte detection in accordance with embodiments of the present technology. In some embodiments, the first nucleic acid 60 and/or the second nucleic acid 70 comprise synthetic (e.g., non-naturally occurring) nucleotide sequences. As shown in FIG. 5, the increased proximity of the first nucleic acid 60 and the second nucleic acid 70 can facilitate hybridization. For example, when proximity is increased between the first nucleic acid 60 and the second nucleic acid 70, orientation of the first nucleic acid 60 with respect to the second nucleic acid 70 and increased relative concentration facilitates hybridization. In addition, to form a hybridized segment 90, the first nucleic acid 60 and the second nucleic acid 70 are in sufficiently close proximity with one another such that one or more nucleotides of the first nucleic acid 60 and one or more nucleotides of the second nucleic acid 70 are complementary and align. For example, the first nucleic acid 60 and the second nucleic acid 70 are hybridized and form a structure where PE-iSDA can initiate. The hybridized segment 90 of the first nucleic acid and the second nucleic acid can be a nucleation site for PE-iSDA.

As illustrated in FIG. 5, the hybridized segment 90 includes overlapping portions of the first nucleic acid 60 and the second nucleic acid 70. For example, the first nucleic acid 60 can include 3 nucleotides in the first overlapping portion. In other embodiments, the first nucleic acid 60 includes 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in the first overlapping portion. In some embodiments, the second nucleic acid 70 includes 3 nucleotides in the second overlapping portion. In other embodiments, the second nucleic acid 70 includes 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in the second overlapping portion.

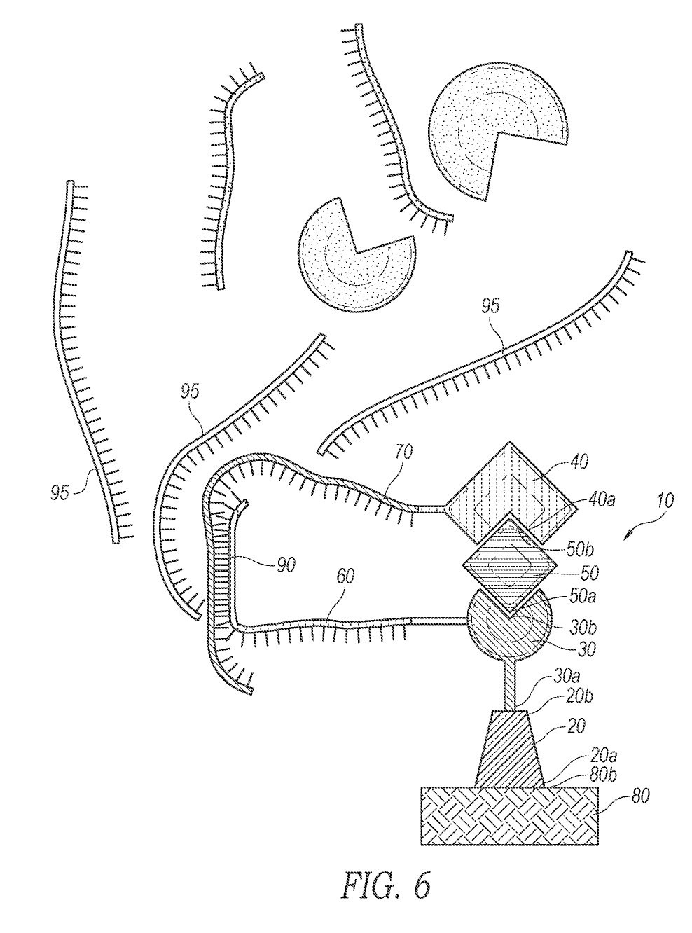

FIG. 6 illustrates a fifth step of the method of proximity-enhanced nucleic acid amplified analyte detection. In some embodiments, the method further include amplifying the hybridized segment 90 of the first nucleic acid 60 and the second nucleic acid 70 to generate a plurality of amplicons 95. By immobilizing the one or more binder peptides, such as the first binder peptide 30, the first nucleic acid 60 and the second nucleic acid 70 are tethered in sufficiently close proximity upon binding of the second binder peptide 40 to the analyte to facilitate amplification. The proximity-enhanced methods can provide for amplification of the hybridized nucleic acids at lower concentrations compared to methods including non-immobilized binder peptides. The lower concentrations can also include a reduced copy number per reaction.

In further embodiments of the methods of proximity-enhanced nucleic acid amplified analyte detection of the present technology, the method further includes combining a plurality of nucleic acid amplification compounds with the complex 10, a first primer, and a second primer (not shown). In some embodiments, the nucleic acid amplification compounds, are configured to generate amplicons 95 from the hybridized portion of the first nucleic acid 60 and the second nucleic acid 70, the first primer, and the second primer. For example, amplification can occur using known methods and compounds and/or reagents. In addition, the reagents in known amplification methods can be configured to perform in accordance with the amplification methods which include PCR, iSDA, PE-iSDA and hybridization chain reaction. Moreover, the methods do not include ligation of a portion of a nucleic acid to a portion of another nucleic acid, or ligation of a nucleotide to another nucleotide.

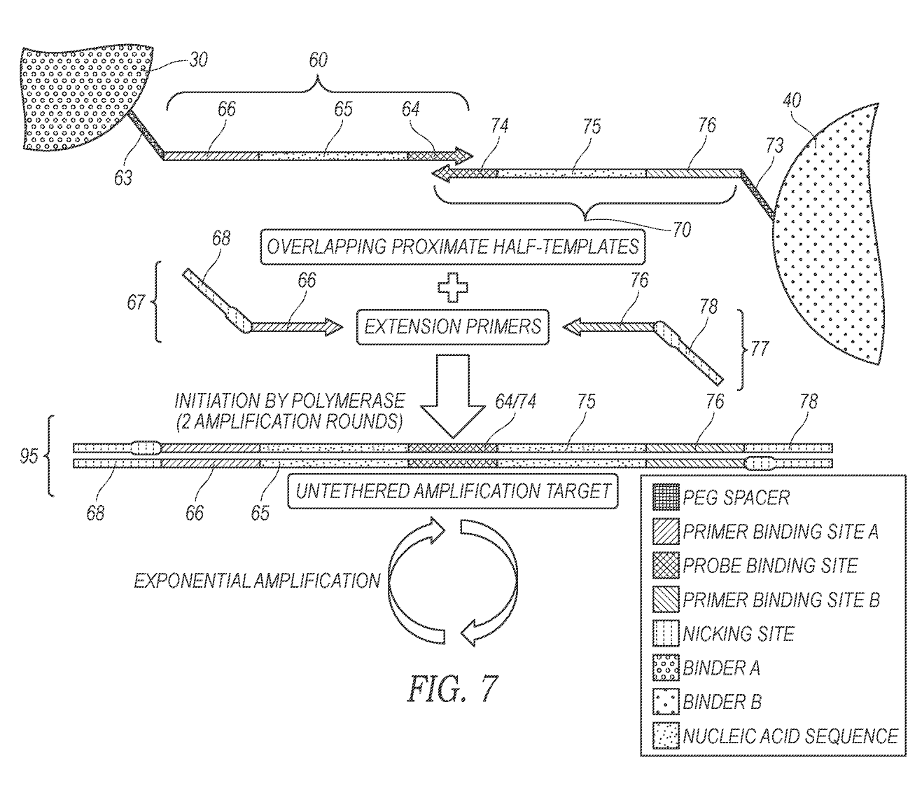

FIG. 7 illustrates one or more nucleic acids associated with the methods of proximity-enhanced nucleic acid amplified analyte detection in accordance with embodiments of the present technology. As illustrated, the methods described herein further include conjugating a first nucleic acid 60 to a first binder peptide 30 (e.g., binder A) and a second nucleic acid 70 to a second binder peptide 40 (e.g. binder B). The first nucleic acid 60 comprises a primer binding site 66 (e.g., primer binding site A) and at least a portion of a probe binding site 64. Similarly, the second nucleic acid 70 comprises a primer binding site 76 (e.g., primer binding site B) and at least a portion of a probe binding site 74. While FIG. 7 illustrates both the first nucleic acid 60 and the second nucleic acid 70 conjugated to binder A 30 and binder B 40, respectively, by a spacer 63/73, it will be appreciated that either the first nucleic acid 60, the second nucleic acid 70, both, or neither can be conjugated to binder A 30 and/or binder B 40 by the spacer 63/73.

By combining features of iSDA with proximity-enhanced detection of analytes (e.g., proteins), the strength of an emitted signal and sensitivity of detecting fewer analytes in a sample are increased. In some embodiments, the sensitivity can be increased to detecting fewer analytes corresponding to the plurality of amplicons. For example, the methods of proximity-enhanced nucleic acid amplified analyte detection can detect about 1×10⁸, about 5×10⁷, about 1×10⁷, about 5×10⁶, about 1×10⁶, about 5×10⁵, about 1×10⁵, about 5×10⁴, about 1×10⁴, about 5×10³, about 1×10³, about 500, about 100, about 50, about 40, about 30, about 20, about 10, about 9, about 8, about 7, about 6, about 5, about 4, about 3, about 2 copies of amplicons, about 1 copy, or one copy of an amplicon.

The first nucleic acid 60 and the second nucleic acid 70 can be proximity enhanced amplification templates, such as a first template and a second template. For example, the first template and the second template each comprise a primer binding site 66/76 and a portion of a probe binding site 64/74. More specifically, the first template comprises a first primer binding site 66 and a first portion of the probe binding site 64 and the second template comprises a second primer binding site 76 and a second portion of the probe binding site 74. In addition, the hybridized segment can be the probe binding site. Both the first template 60 and the second template 70 each include a nucleic acid sequence, such as a first nucleic acid sequence 65 and a second nucleic acid sequence 75, respectively.

In some embodiments, the first nucleic acid 60 and/or the second nucleic acid 70 are templates, such as a first template and a second template, respectively. The first template 60 and/or the second template 70 can include, for example, a 3′ overhang and at least one nicking site. In addition, the 3′ overhangs of the first template 60 and the second template 70 can be reverse complementary. (Not shown.) The disclosed method can further include hybridizing a segment of the first nucleic acid 64 with a segment of the second nucleic acid 74.

The one or more nucleic acids associated with the method can further include two extension primers 67/77 included in amplification, with each extension primer binding to a primer binding site of the first nucleic acid or of the second nucleic acid, such as a first primer binding site 66 and a second primer binding site 76. In some embodiments, the extension primers include nicking sites 68/78. When combined with nicking endonuclease, polymerase, nucleotides, and additional compounds associated with a polymerase chain reaction, the extension primers 67/77 amplify at least a portion of the first nucleic acid 60 and at least a portion of the second nucleic acid 70 upon binding of the extension primers 67/77 of the corresponding primer binding sites 66/76.

As illustrated in FIG. 7, amplification can be initiated by polymerase, which can generate an amplification target, such as an untethered amplification target. In some embodiments, the methods include generating the plurality of amplicons by exponential amplification, for example, at about 40° C., about 45° C., about 46° C., about 47° C., about 48° C., about 49° C., about 50° C., about 51° C., about 52° C., about 53° C., about 54° C., about 55° C., about 60° C., or about 65° C. The plurality of amplicons can be generated in about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, or about 20 minutes.

In some embodiments, the detecting step further includes combining a plurality of nucleic acid detection compounds with the amplicons and/or introducing the detection compounds prior to amplification and/or during amplification. The detection compounds are configured to produce a signal when bound to and/or incorporated within the amplicons. In addition, method can further include the step of quantifying an amount of the analyte present in the sample based on a number of generated and detected amplicons (not shown).

### III. EXAMPLES

Several aspects of the present technology are set forth in the following examples.

### III.A. Example 1: Generation of Binder Pairs for Influenza NP And HA, and RSV F Protein.

Protein binder pairs for influenza nucleoprotein (“NP”) and hemagglutinin (“HA”) and protein binder pairs for respiratory syncytial virus (“RSV”) protein F will be generated. One of the proteins from each of the NP, HA, and RSV protein binder pairs will be optimized to bind to protein analytes, such as NP, HA, and RSV proteins respectively, with high affinity and specificity at complementary epitopes. In addition, the other protein from each of the NP, HA, and RSV protein binder pairs will be optimized to bind to one or more porous material surfaces. Finally, both proteins of the NP, HA, and RSV protein binder pairs will be designed for conjugation to oligonucleotides at precisely-defined amino acids. These amino acids will be selected for compatibility with PE-iSDA.

Three strategies will be used to generate protein binder pairs, (1) adapting commercially available antibody pairs that bind an analyte of interest, (2) modifying existing computationally designed mini-protein binder pairs that bind an analyte of interest, and (3) de novo computational design to produce new mini-protein binder pairs that bind an analyte of interest. The NP binder pairs will be generated using strategy (1), the HA binder pairs will be generated using strategy (2), and the RSV binder pairs will be generated using strategy (3).

The NP binder pairs will be designed by modifying a pair of commercially available antibodies that is specific to complementary epitopes of the influenza NP protein. The NP antibodies will be modified to support PE-iSDA by conjugating adapter oligonucleotides, such as those from a commercially-available proximity ligation assay method and components (e.g., kit). Each of the adapters will be hybridized to half of an iSDA template.

The HA binder pairs will be designed by modifying known HA stem binders and homo-trimeric HA head binders having less than nanomolar (nM) binding affinities for the HA protein. The HA stem binder can bind to either Group I influenza strains, Group II influenza strains, and/or engaging a sialic binding site of other strains. In addition, protein design may be used to engineer HA homo-oligomeric trimer sialic acid site binders having close to atomic level accuracy to bind a trimeric HA with low off rates. Non-overlapping pairs of mini-protein binders will also be designed for HA Groups I and II using similar strategies.

The RSV binder pairs will be designed using high resolution crystal structures of the pre-fusion F (“Pre-F”) and post-fusion F (“Post-F”) proteins bound to neutralizing monoclonal antibodies at different sites. The Post-F conformation will be a diagnostic target because many of the F proteins are in the Post-F conformation. In addition, non-overlapping binders that may target both the Pre-F and Post-F conformations, for example the 101F and motavizumab mAbs binding sites, will also be designed.

To achieve de novo computational design of binder proteins, low energy structures and sequences for thousands of candidate binding proteins will be developed, synthetic DNA libraries encoding these candidate proteins will be generated, and candidate proteins will be expressed on surfaces of yeast cells. To identify the most stable designed binders, libraries of yeast cells expressing the yeast cells expressing the candidates will be incubated at a range of temperatures (e.g., about 25° C. to about 60° C.) and with increasing concentrations of serum proteases. Next generation DNA sequencing will be used to identify the most stable designs and yeast cells expressing these designs will be sorted.

Libraries of sorted cells displaying protease resistant (e.g., folded) proteins will be expanded and selected for further functional testing for binding with high affinity to target proteins of interest. Once yeast clones for individual protein binder designs have been identified, they will be individually expressed on the surface of single clones of yeast. Ligand affinities will be determined using different concentrations of fluorophore-labeled target proteins (e.g. HA or RSV Post-F). Subsequent rounds of mini-protein affinity maturation will be achieved using saturation site mutagenesis. From this, optimized His6 tagged protein binders will be generated using E. coli-based recombinant protein production techniques. His6 tagged proteins will be purified using immobilized metal chelate affinity (IMAC) purification and tested for coincident target binding using biolayer interferometry. The binding mode and structures of optimized protein binders will be verified using X-ray crystal structure determination.

Optimized protein binders will undergo additional engineering for compatibility with PD-iSDA, such as (1) modifications to achieve high affinity binding to a substrate (e.g., a fibrous paper substrate used in the 2DPN assay system), and (2) covalent conjugation of PD-iSDA oligonucleotide pairs. Clones of individual yeast strains expressing the optimized protein binders will be produced in E. coli or yeast as secreted proteins having a tag (e.g., Tev-GSL-His6-SpyCatcher) fused to their C-termini. “Tev” is a site-specific tobacco etch virus protease cleavage site (e.g., Tev site ENLYFQ/G where Q is left on C-term) that allows for cleavage and removal of the tag. GSL is a variable length flexible Glycine-Serine linker that will be used to adjust the optimized binder for PD-iSDA applications. The His6 tag will be used for IMAC purification as described above. The AviTag sequence may be biotinylated using biotin ligase (e.g, AviTag™ GLNDIFEAQKIEWHE). SpyCatcher is a small protein that covalently attaches to a 13-amino acid SpyTag peptide which will be attached to the oligonucleotides.

In addition, single Cys residue modifications may be introduced into the optimized protein binders using maleimide-thiol coupling chemistry for efficient syntheses of oligonucleotide-binder conjugates. Mini-protein binders having less than about 40 amino acids may be purchased as synthetic peptides from CSbio or Genescript with a Cys residue for coupling to PE-iSDA oligonucleotides. The tagged and optimized protein binders will be purified on a Ni-NTA Sepharose column using the His6 tag. Fractions containing tagged and optimized protein binders will be verified using SDS-PAGE, concentrated, and purified using size exclusion chromatography (e.g., Superdex 200). These tagged and optimized protein binders may be complexed with one or more binder proteins, such as an immobilizer homo-trimeric protein comprised of SpyCatcher quenched with SpyTag, a peptide aldolase homo-trimer, and monomeric streptavidin. The immobilizer protein binds with high affinity to different substrates, such as fibrous papers (e.g., FF08HP, Fusion 5, and GF8964). The immobilizer may be used to anchor one or more proteins having a biotin Avitag, or SpyTag (if the SpyCatcher has not been quenched).

### III.B. Example 2: Evaluation of Binder Pair Candidates to Support PE-iSDA.

Optimized binder pairs will be tested for the following, (1) ability to conjugate PE-iSDA oligonucleotide adapters to conjugation sites on each of the binder proteins, (2) ability of one of the binder proteins of the pair to immobilize to a substrate (e.g., a porous material) or bind to an immobilizer, and (3) ability of each binder of the pair to bind to complementary sites on the target analyte.

Optimized binder pairs will be conjugated to SpyTag-iSDA oligo adapters with or without spacers (e.g., polyethylene glycol “PEG”) and validation will be performed using Ellman's assay. Immobilization of one binder of the pair, such as the capture binder, will be tested under flow conditions using detergent solutions and a visible protein stain. Binding of the optimized binder pairs to the analyze will also be characterized. Optimized binder pairs conjugated to oligonucleotides which fail any of these tests will be re-evaluated for one or more of the following, alternative oligonucleotide design, alternative oligonucleotide conjugation chemistries that may be used, or alternative design of binder conjugation sites.

Biolayer interferometry (BLI) experiments will be performed to ensure that desired optimized protein binder pairs are capable of simultaneous and non-competitive target binding. In BLI experiments, Avi tag biotin labeled recombinant target proteins (e.g., NP, HA of Group I or II, and RSV Pre-F or Post-F proteins) will be loaded onto streptavidin BLI probes that will be sequentially exposed to one of the proteins of the protein binder pair (e.g., binder 1) followed by washing and addition of binder 2. Protein binder pairs capable of simultaneously binding to the target are expected to have a clear signature of increased BLI signal for simultaneous binding of both binder 1 and 2.

### III.C. Example 3: Demonstration of PE-iSDA Using Binder Pairs.

Binder pairs that have passed tests for conjugation, immobilization, and target binding functionalities, will be tested for their ability to support amplification from associated PE-iSDA template halves and will establish PE-iSDA functionality using the binder pairs, purified target analyte, and template strands complementary to the oligo adapters at their 5′ ends and to each other at their 3′ ends. Amplification from binder pairs will be tested individually and in combination with the target protein (e.g., analyte). Individual amplification tests using the protein binder will be performed in tube with a high concentration of untethered complementary half-template. Real-time measurements will be made using a fluorescent probe.

In addition, oligonucleotide template halves may also be conjugated directed to the binders rather than using adapters. Combined binder-supported amplification tests will be performed in tube with both binder-adapted PE-iSDA template halves in the presence and absence of the analyte. The capture binders may also be immobilized in microplate wells, comparable to an ELISA format. Target analyte (or a non-target control) will be added, incubated, and washed. The detection binders will also be added, incubated, and washed. The amplification reagents will then be added and amplification will be measured in real time using a fluorescent probe. The overlap sequence of template halves may also be modified to match a free energy of hybridization under PE-iSDA conditions. Finally, successful half-template sequences will be directly conjugated to binders and re-tested for use in substrate-based (e.g. paper) and in other assay systems.

### III.D. Example 4: Demonstration of PE-iSDA Using Binder Pairs.

To increase the stringency of the amplification portion of PE-iSDA, oligonucleotide template halves, which hybridize to one another for amplification to occur, were designed with a 10 bp hybridization overlap. A fluorescent signal is observed upon hybridization-dependent amplification when the hybridization region is positioned within a DNA sequence where fluorescent probe(s) bind. In addition, because the hybridization reaction between two single stranded DNA molecules is concentration dependent, the concentration at which hybridization occurs is reduced when the template halves are brought within sufficiently close proximity, such as through conjugation to proteins (e.g., biotin) which bind other proteins (e.g., streptavidin).

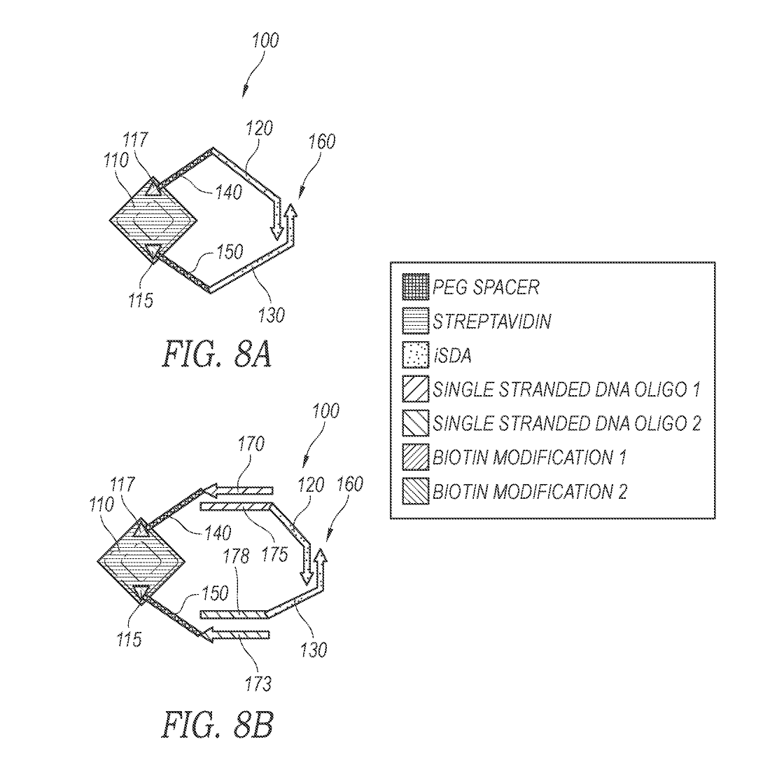

FIG. 8A illustrates a PE-iSDA reaction complex 100 using a conjugation protein 110. 60 bp single stranded DNA template halves 120/130 were designed based on the Neisseria gonorrhoeae SSDM gene and synthesized with biotin conjugates 115/117 at their 5′ ends. Biotinylated templates 120/130 are bound to streptavidin 110 which brings biotinylated PE-iSDA templates 120/130 into sufficiently close proximity to one another. As shown, streptavidin 110 is bound to two biotin molecules 115/117, however because streptavidin 110 comprises four biotin binding sites, up to two additional biotin molecules and oligos may be bound to a single streptavidin molecule.

Additional template halves were tested using a PEG spacer system. As illustrated in FIG. 8A, oligo adapters 170/173 were each modified with a PEG spacer 140/150 and biotin 115/117. These adapters 170/173 contain a 25 bp sequence homologous to an adapter sequence on specific oligonucleotide template halves 175/178. To obtain a 1:1:1:1:1 ratio of streptavidin: adapter 1:adapted template 1:adapter 2:adapter template 2, two adapter sequences were used 170/173, one binding to the first template half 175 of the system, and one which binds to the second template half 178 of the system.

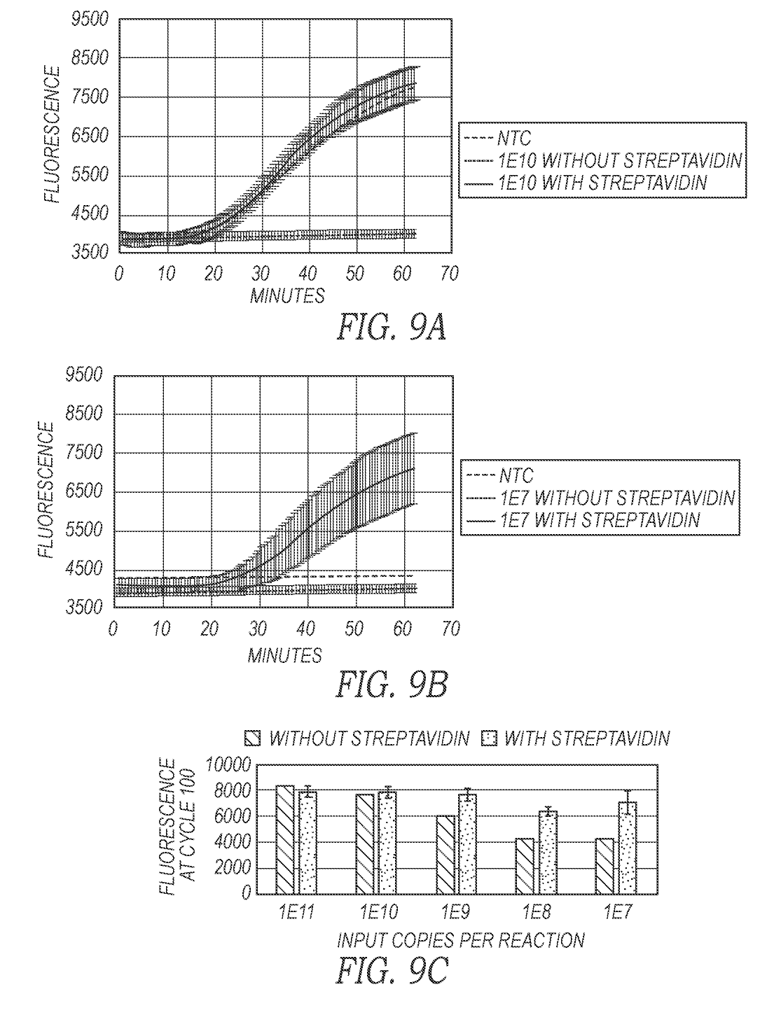

FIGS. 9A and 9B are graphs displaying amplification results of the PE-iSDA reaction illustrated in FIG. 8A where amplification occurred from directly biotinylated template halves in the presence and absence of streptavidin. For FIG. 9A, the input copy number was 1×10¹⁰ per reaction. For FIG. 9B, the input copy number was 1×10⁷ per reaction. FIG. 9C illustrates the average fluorescent signal obtained during the final measurement of each amplification reaction using a variety of different input copy numbers in the presence and absence of streptavidin. n=1 without streptavidin and n=2-3 with streptavidin condition, n=2-3. The thin bars of the with streptavidin condition represent standard error of the mean. Using a high copy number, amplification in the presence and absence of streptavidin appears substantially similar (FIG. 9A and 9C). However, at lower copy numbers, there was a difference in amplification. (FIGS. 9B and 9C). This difference was observed following measurement of higher levels of fluorescent signal during the latter half of the amplification reaction.

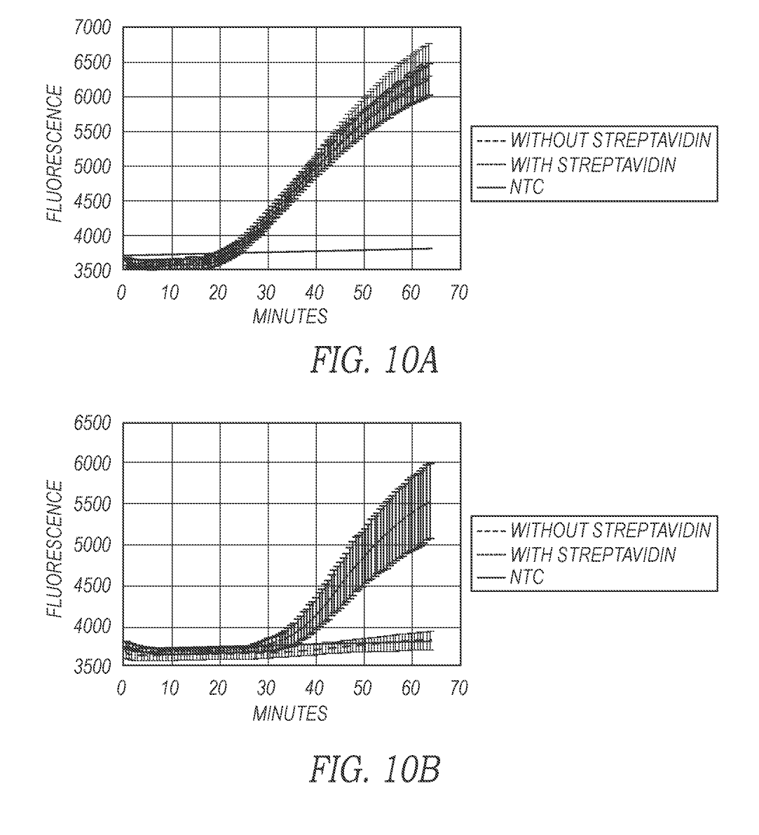

FIGS. 10A and 10B are graphs displaying amplification results of the PE-iSDA reaction illustrated in FIG. 8B. Amplification was measured by increase in fluorescence over time. At a high copy number, such as 1×10¹¹ copies per reaction (FIG. 10A), there was no significant difference in amplification between conditions including streptavidin compared to those in the absence of streptavidin. At a lower copy number, such as 1×10⁷ copies per reaction (FIG. 10B), there is a statistically significant (p<0.05) difference between the two conditions starting at approximately 46 minutes. Values shown are averages of at least three experiments. Thin bars represent standard error of the mean.

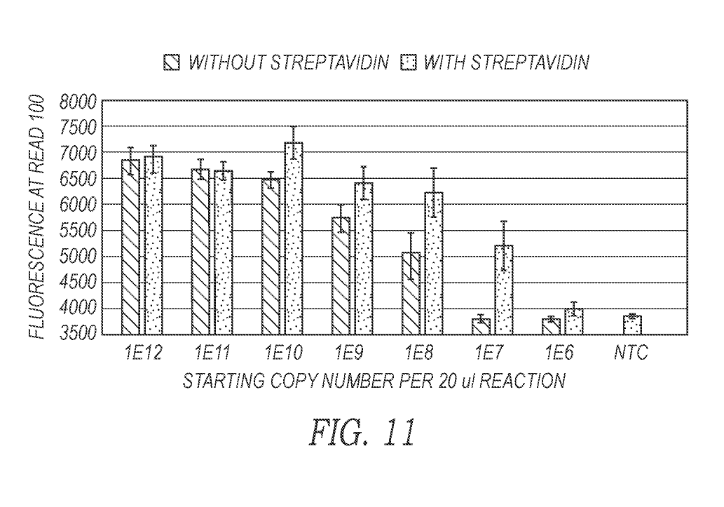

The strength of the fluorescent signal during the last final measurement of each amplification reaction is shown in FIG. 11 as a measure of amplicon number after amplification. Thick bars represent the average of at least three experimental values. Thin bars represent the standard error of the mean. There is a statistically significant difference (p<0.05) between conditions including streptavidin compared to those in the absence of streptavidin when the starting copy number of each of the components was 1×10⁹ and 1×10⁷ (FIG. 11). The same trend was observed with the 1×10⁸ copy number condition, but variability in those samples was higher than in the other copy number conditions, and the difference was not statistically significant.

The proximity dependent enhanced iSDA, and trends observed with respect to amplification were similar to those obtained using a mecA iSDA assay. The fluorescent probe for mecA amplicons comprises a minor groove binder (MGB) element which increases the kinetics of probe binding compared to the molecular beacon probe used for the SSDM target described above. The MGB probe also has a lower background fluorescence, which aids in distinguishing amplification of a smaller number of amplicons from background noise. As with the SSDM template halves described above, the template halves for mecA iSDA included the hybridization site within the probe binding region. For mecA iSDA, the hybridization length was also 10 bp.

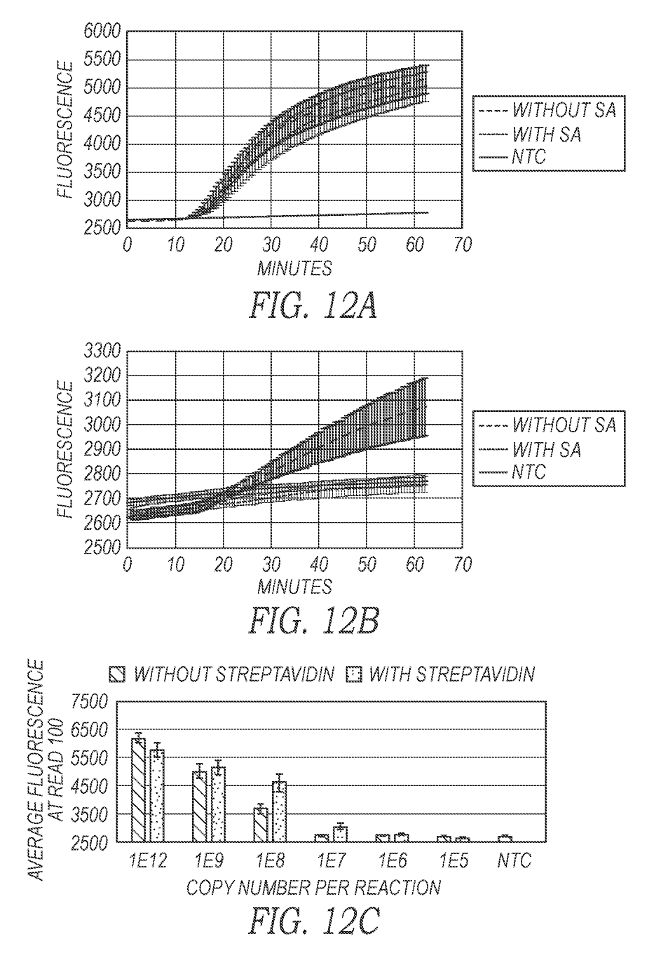

The mecA template halves were biotinylated and included PEG adapter sequences, similar to those illustrated in FIG. 8B. For example, as illustrated in FIGS. 12A-12C, the addition of streptavidin and biotinylated adapters increased proximity between the template halves, thereby increasing the effective concentration of the mecA template halves and decreasing the copy number at which amplification was observed. The mecA system followed a similar pattern as SSDM, described above. Amplification was increased at lower copy number in the presence of streptavidin than in its absence. In the mecA system, amplification rose above baseline fluorescence at 16-20 minutes, rather than at 30-40 minutes as was observed in SSDM. (See FIGS. 9A-11). Due to decreased background fluorescence and increased probe binding kinetics, the time to detection of amplification was decreased. Moreover, in the mecA system, there was a statistically significant increase in fluorescence at the final amplification in the presence of streptavidin in the 1×10⁷ and 1×10⁸ copy number conditions (FIG. 12C). All values shown in FIGS. 12A-12C are the average of four independent experiments. Thin bars represent standard error of the mean.

### IV. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded.

From the foregoing, it will be appreciated that specific embodiments of the technology have been described herein for purposes of illustration, but that various modifications may be made without deviating from the technology. Additionally, other embodiments of the present technology can have different steps, ordering of steps, components, features, and/or procedures than those described herein. For example, other embodiments can include additional elements and features beyond those described herein, or other embodiments may not include several of the elements and features shown and described herein. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A method for detecting an analyte in a sample, the method comprising:
attaching an immobilizer peptide to a substrate;
generating a complex at the immobilizer peptide, the complex including—
a first binder peptide and a second binder peptide, wherein the first binder peptide is bound to the immobilizer peptide,
a first nucleic acid and a second nucleic acid, wherein the first nucleic acid is conjugated to the first binder peptide and the second nucleic acid is conjugated to the second binder peptide, and
the analyte,
hybridizing a segment of the first nucleic acid with a segment of the second nucleic acid;
amplifying the hybridized segment of the first nucleic acid and the second nucleic acid to generate a plurality of amplicons; and
detecting one or more of the plurality of amplicons.

2. The method of claim 1, further comprising:
based on a number of generated amplicons, quantifying an amount of the analyte present in the sample.

3. The method of claim 1 wherein the complex is generated by the first binder peptide binding to a first region of the analyte and a second binder peptide binding to a second region of the analyte.

4. The method of claim 1 wherein the first nucleic acid is conjugated to the first binder peptide by a first spacer at a first conjugation site and the second nucleic acid is conjugated to the second binder peptide by a second spacer at a second conjugation site.

5. The method of claim 4 wherein the first spacer and the second spacer are PEG spacers.

6. The method of claim 1 wherein the first binder peptide is a capture binder.

7. The method of claim 1 wherein the second binder peptide is a detection binder.

8. The method of claim 1 wherein the portion of the first nucleic acid and the portion of the second nucleic acid which hybridize are reverse complementary.

9. The method of claim 8 wherein the segment of the first nucleic acid that hybridizes with the second nucleic acid comprises two nucleotides, three nucleotides, four nucleotides, or five nucleotides, and wherein the segment of the second nucleic acid that hybridizes with the first nucleic acid comprises two nucleotides, three nucleotides, four nucleotides, or five nucleotides.

10. The method of claim 1 wherein the segments of the first nucleic acid and second nucleic acid that hybridize is a probe binding site.

11. The method of claim 1 wherein the first nucleic acid comprises a first primer binding site which binds a first primer and wherein the second nucleic acid comprises a second primer binding site which binds a second primer.

12. The method of claim 11 wherein the first primer comprises a first nicking site and wherein the second primer comprises a second nicking site.

13. The method of claim 12 wherein the complex further comprises the first primer and the second primer.

14. The method of claim 13 wherein amplifying further comprises combining a plurality of nucleic acid amplification compounds with the complex, the first primer, and the second primer, are configured to generate amplicons from the hybridized portion of the first nucleic acid and the second nucleic acid, the first primer, and the second primer.

15. The method of claim 14 wherein detecting further comprises combining a plurality of nucleic acid detection compounds with the amplicons, the detection compounds configured to produce a signal when combined with the amplicons.

16. A method for generating an immobilized analyte detection complex, the method comprising,
attaching an immobilizer peptide to a solid substrate;
generating a complex at the immobilizer peptide, further comprising
binding a first portion of a first binder peptide to a first region of an analyte,
binding a second portion of a second binder peptide to a second region of the analyte, and
binding a section portion of the first binder peptide to a first zone of the immobilizer peptide.

17. The method of claim 16 wherein the first binder peptide is a capture binder, and wherein the second binder peptide is a detection binder.

18. An immobilized analyte detection composition, comprising,
an immobilizer having a first zone and a second zone, the first zone configured to bind to a substrate;
a first binder peptide having a first portion and a second portion, the first portion configured to bind to the second zone of the immobilizer and the second portion configured to bind to a first region of the analyte; and
a second binder peptide configured to bind to a second region of the analyte.

19. The immobilized analyte detection composition of claim 19 wherein the first binder peptide is an antibody, an antibody fragment, or an aptamer, and wherein the second binder peptide is an antibody, an antibody fragment, or an aptamer.

20. The immobilized analyte detection composition of claim 20 wherein the antibody, antibody fragment, or the aptamer, is an engineered peptide, a designer peptide, a synthetic peptide, or a combination thereof.
